(19) United States
(12) Patent Application Publication
(10) Pub. No.: US 2014/0221781 A1
(43) Pub. Date: Aug. 7, 2014

(54) Method and Device for Monitoring at Least One Vehicle Occupant and Method for Operating at Least One Assistance Device

(71) Applicants: Michael Schrauf, Esslingen (DE); Michael Simon, Ulm (DE); Galia Weidl, Boeblingen (DE); Ralf Herrtwich, Gerlingen (DE); Ulrich Kressel, Ulm (DE)

(72) Inventors: Michael Schrauf, Esslingen (DE); Michael Simon, Ulm (DE); Galia Weidl, Boeblingen (DE); Ralf Herrtwich, Gerlingen (DE); Ulrich Kressel, Ulm (DE)

(73) Assignee: Daimler AG, Stuttgart (DE)

(21) Appl. No.: 14/238,922

(22) PCT Filed: Jul. 25, 2012
(86) PCT No.: PCT/EP2012/003145

§ 371 Date: Apr. 3, 2014

(30) Foreign Application Priority Data
Aug. 17, 2011 (DE) 10 2011 110 486.4

(51) Int. Cl. A61B 5/18; A61B 5/0205; A61B 5/00; A61B 5/1455; A61B 5/01; A61B 5/029; A61B 5/026; G06K 9/00; A61B 5/16

(52) U.S. Cl. CPC A61B 5/18; G06K 9/00832; A61B 5/02055; A61B 5/7278; A61B 5/165; A61B 5/01; A61B 5/029; A61B 5/0261; A61B 5/4824; A61B 5/14551; A61B 5/7257; A61B 5/7282; A61B 5/742; A61B 5/7405; A61B 5/7455; A61B 5/746; A61B 5/747; A61B 5/02416
USPC 600/301

(57) ABSTRACT

A method and a device for monitoring at least one vehicle passenger in a vehicle involve capturing images of the vehicle passenger by an image capturing unit and analyzing the captured images using an image processing unit. Vital parameters of the vehicle passenger are determined by the image analysis of the captured images. The vital parameters can include a pulse rate, a breathing rate and a breathing volume, a heart rate variability is determined from these vital parameters.

### BACKGROUND AND SUMMARY OF THE INVENTION

Exemplary embodiments of the present invention relate to a method for monitoring at least one vehicle passenger, a device for monitoring at least one vehicle passenger, and a method for controlling at least one assistance device.

European patent document EP 1 182 089 B2 discloses a method to warn a driver of a vehicle when a critical situation is detected by vehicle sensors. Furthermore, an alertness level of the driver is determined, wherein an image of the driver is captured by a camera device and the image is processed by a processing unit. An alertness level of the driver is determined from the image, wherein a line of vision of the driver is determined by the processing unit. In the case of a determined high alertness level of the driver, the task of warning is dispensed with. In the case of several warnings to be emitted simultaneously, a first warning of a critical situation, which lies in the line of vision of the driver, is only emitted after a second warning of a critical situation, which lies outside of the line of vision of the driver.

German patent document DE 10 2009 016 936 A1, assigned to the assignee of the present application, the complete content of which is hereby included by reference, discloses a driving assistance system for supporting a driver of a vehicle in the case of fatigue. The driving assistance system comprises a drowsiness detection system to detect fatigue of the driver. When fatigue of the driver is detected by means of the drowsiness detection system, a flashing warning light of the vehicle is activated to warn other road users.

Furthermore, in the article by Ming-Zher Poh, Daniel J. McDuff, and Rosalind W. Picard, “Noncontact, automated cardiac pulse measurements using video imagine and blind source separation,” Opt. Express 18, 10762-10774 (2010), the complete content of which is hereby included by reference, a method is described in which essentially the heart rate of the people is determined by means of video sequences, captured by low-resolution video cameras under normal lighting conditions, of faces of people. Therein the difference in brightness of the light reflected by the skin is measured and analyzed. First, the positions of the faces in the visual field of the camera are identified and the video image in this section is broken up into red, green and blue portions and analyzed. During a heartbeat the blood vessels—above all the arteries—expand slightly, as the pressure increases. This expansion causes an increase of the optical absorption and therefore also a decrease in the intensity of the light that is reflected by the face.

Exemplary embodiments of the present invention are directed to an improved method and an improved device to monitor at least one vehicle passenger in a vehicle and an improved method to control at least one assistance device of a vehicle.

In accordance with the method of the present invention, monitoring at least one vehicle passenger in a vehicle involves capturing images of the vehicle passenger by means of at least one image capturing unit and analyzed by means of an image processing unit.

According to the invention, vital parameters of the vehicle passenger are determined by the image analysis of the captured images, wherein a pulse rate, a breathing rate and a breathing volume are determined as vital parameters and heart rate variability is determined therefrom. The heart rate variability, also referred to as the HRV, is connected to the heart rate and the breathing i.e. the breathing rate and the breathing volume via the respiratory sinus arrhythmia, which describes a fluctuation of the heart rate in synchronization with the breathing. Additionally vital functions or vital signs or health risks such as resilience, stress tolerance, a cardiovascular risk and further health risks as well as positive and negative emotional states such as joy, stress or anger can be determined from the heart rate variability.

The determination of the vital parameters enables a vital state of the vehicle passenger, for example of a vehicle driver of the vehicle, to be assessed and suitable measures to be initiated in the case of a determined deterioration of the vital state, for example to activate functions of the assistance device of the vehicle, in order to at least indicate a potential risk of accident or if necessary to prevent an accident, to request help and/or to increase the comfort of the vehicle passenger(s), in order to improve the vital state. This means the assistance device can, for example, be adapted to a current vehicle passenger state or to features of the vehicle driver, wherein the features thereof can be evaluated by means of a stress level or the emotional state of the vehicle driver.

Therein, an image capturing unit that is already installed in the vehicle, for example in the region of a steering column, and is directed toward the vehicle passenger(s), preferably toward the vehicle driver, serves as an image capturing unit. The vital parameters of the respective vehicle passenger are all detected simultaneously by means of this image capturing unit or all data is detected simultaneously, from which the vital parameters of the respective vehicle passenger can be determined by means of an image analysis. Such image capturing units are installed in the vehicle, for example to recognize alertness of the vehicle driver. To carry out the method, a video camera with low resolution is sufficient therein, for example a so-called CCD camera. Special lighting devices are also not required, however environmental lighting, for example daylight, is already sufficient. A robustness of a signal detection of the image capturing unit is clearly improved by partially redundant information, for example in a RGB color signal of the image capturing unit, and in particular by the possibility of the signal detection with a black and white camera for dark driving sections, i.e. for a dark environment, for example during a drive through a tunnel or at night. Therein, a signal detection in the so-called HSC black and white signal occurs, if necessary by means of lighting by an infra-red light source. Therein, for example, a separate image capturing unit is not required, but the present image capturing unit is switched to a black and white mode. Advantageously, this occurs automatically depending on respective proportions of environmental light.

Advantageously, in particular a skin temperature and/or pumping strength of the heart and/or a blood pressure and/or an oxygen concentration in the blood of the vehicle passenger, also referred to as an oxygen saturation, and/or autonomous bodily functions, in particular functions of an autonomous nervous system of the vehicle passenger and/or pain states of the vehicle passenger can be determined as further vital parameters. For example, further diagnostic parameters are detected or recognized by means of the vital parameters, for example tachycardia, brachycardia and/or viral infections, by means of the heart rate variability, breathing, i.e. the breathing rate and/or the breathing volume, and/or the skin temperature. An assessment of the current vital state of the vehicle passenger can be optimized by means of these further vital parameters, such that if necessary, for example inattention of the vehicle passenger, for example of the vehicle driver, or a threatening or already occurring unconsciousness or severe health problems can be indicated.

Advantageously, the determined vital parameters are recorded and stored in the vehicle, such that they are available, for example, in an emergency situation during medical care. They can, for example be transferred together with an automatically initiated emergency call to a hospital or casualty center, such that quick and adequate help can be initiated.

Preferably, the vital parameters are determined by a detection of an optical absorption value and/or an intensity value of reflected light of a skin region of the vehicle passenger and/or by a determination of a change of this optical absorption value and/or of this intensity value. During a heartbeat, blood vessels, in particular arteries, expand slightly, as pressure increases in the blood vessels. This expansion causes an increase of the optical absorption and therefore also a decrease of the intensity of reflected light that is reflected by the skin region. By determining the differences in brightness of the light reflected by the skin region over time, vital parameters, in particular the pulse rate, breathing rate and breathing volume can be determined. This method is also called photoplethysmography. The foundations of the method are, for example, described in the article by Ming-Zher Poh, Daniel J. McDuff, and Rosalind W. Picard, “Noncontact, automated cardiac pulse measurements using video imaging and blind source separation,” Opt. Express 18 10762-10774 (2010), the complete content of which is hereby included by reference.

Preferably at least one region suitable for image analysis is searched for in each of the images during the image processing and only this region is analyzed. This region is a region in the images, in which a sufficiently large skin region of the vehicle passenger is displayed. Preferably a region of the images is searched for and analyzed as a region suitable for image analysis, which contains the face of the vehicle passenger, as in this region a sufficiently larger skin region that can be analyzed is available and as, in particular, image capturing units that are already installed in the vehicle and which are used, for example, to recognize alertness, are directed towards the face of the vehicle passenger, in particular of the vehicle driver. One or several vital parameters of several vehicle passengers can be detected simultaneously with the method, wherein the image region is determined, in which the faces of the vehicle passengers are situated and are analyzed separately from one another.

Advantageously, color images are captured as images of the vehicle passengers, in particular so-called RGB images, wherein the region of the images suitable for image analysis is split into individual color channels and at least one color channel is analyzed to determine the at least one vital parameter. For example, the region suitable for image analysis is divided into red, green and blue portions, i.e. color channels. Therein, preferably, the green portion or color channel is analyzed, as the accuracy of the obtained results is highest with this channel.

The skin temperature, the oxygen concentration or oxygen saturation and the blood pressure of the vehicle passenger can be determined from a combination of the color channels, more exactly from a phase shift and/or a change in amplitude between the color channels or color signals.

Preferably, a spectral power density of the color channel of the region suitable for image analysis is determined by means of the fast Fourier transform. The difference in brightness of the light reflected by the skin region over time can then be determined by means of the determined spectral power density and the vital parameter(s) can be determined therefrom.

The vital parameters determined in this way can also be combined with values of the vehicle passengers determined in another way, in order to thus obtain a better overall image of the state of the vehicle passenger. In this way, for example, fatigue or inattention of the vehicle driver can be determined in good time and with a low error rate. Thus, for example, movements of the respective vehicle passenger, in particular of the vehicle driver, can be determined by sensors in the vehicle seat, which can indicate, restlessness, stress or the beginnings of fatigue. This can, for example, be combined with the determined pulse rate and/or with other determined vital parameters, in order to obtain a better estimate.

In the case of a device to monitor at least one vehicle passenger in a vehicle, in particular to carry out the method to monitor at least one vehicle passenger in a vehicle, images of the vehicle passenger are able to be captured by means of at least one image capturing unit and are able to be analyzed by means of an image processing unit. According to the invention, vital parameters of the vehicle passenger are determined by the image analysis of the captured images by means of the image capturing unit, wherein a pulse rate, a breathing rate and a breathing volume are determined as vital parameters and a heart rate variability is determined therefrom. This enables a vital state of the vehicle passenger, for example a vehicle driver of the vehicle to be assessed and suitable measures to be initiated in the case of a determined deterioration of the vital state, for example functions of an assistance device of the vehicle to be activated in order to at least indicate a potential risk of accident or if necessary to prevent an accident, to request help and/or to increase the comfort of the vehicle passenger(s) in order to improve the vital state, as has already been described with regard to the method, which is able to be carried out by means of the device, to monitor at least one vehicle passenger in a vehicle.

In a method to control at least one assistance device of a vehicle using the method to monitor at least one vehicle passenger, according to the invention, at least one function of the assistance device of the vehicle is activated if at least one vital parameter deviates from a predetermined value or value region for this vital parameter. In this way, the assistance device can intervene in a supporting manner in the case of poor or deteriorating vital parameters of the vehicle passenger, in particular of the vehicle driver, in order indicate, for example a potential accident or, if necessary, to prevent an accident, to request help and/or to increase comfort of the vehicle passengers(s) in order to thus improve the vital state.

Preferably, an intervention in a steering device, a braking device and/or a drive train of the vehicle and/or an optical, aural and/or haptic warning device is activated as a function of the assistance device. In this way, the vehicle, as for example, is described in German patent document DE 10 2009 016 936 A1, can be steered to the edge of a road and brought to a standstill there by a corresponding intervention in the steering device, braking device and/or drive train of the vehicle. Particularly advantageously the intervention in the steering device, braking device and/or in the drive train of the vehicle can occur in such a way that the vehicle is steered autonomously to the nearest emergency assistance establishment, for example to the casualty of the nearest hospital.

Alternatively or additionally, warning devices of the vehicle can be activated, for example outer warning devices in the form of flashing warning lights and/or a horn of the vehicle, in order to warn other road users, and/or warning devices within the vehicle can be activated, in order to warn the vehicle passenger(s), for example in order to wake a vehicle driver who has fallen asleep or to indicate to other vehicle passengers a poor or deteriorating vital state of the vehicle driver such that they can operate the steering wheel and/or the handbrake of the vehicle or these are actuated by actuators in order to brake the vehicle and/or to steer the vehicle to the edge of the road.

Advantageously, at least one comfort function and/or emergency call function is activated, alternatively or additionally, as a function of the assistance device. Through the activation of the comfort function or a plurality of such comfort functions, vital parameters of the vehicle passenger(s) can be improved, for example if the detected vital parameter(s) indicated a fatigue in particular of the vehicle driver. For example, a massage function of a vehicle seat, an interior ventilation and/or an air conditioning function of the vehicle can then be activated. Therein for example, essential oils or another refreshing medium can be introduced into the interior of the vehicle to refresh the vehicle passengers. Furthermore, the relevant vehicle passenger can be informed about his current vital situation by optical, aural or haptic means, preferably with a recommendation of suitable counter measures, for example take a restorative break, to move regularly and/or to take on sufficient fluids. In this way, the vehicle passenger can also be reminded, for example, to take required medication. Furthermore, devices of the vehicle to support or calm the driver can be targetedly activated, for example in an increased stress level recognized by means of the vital parameters, for example a navigation device or audio equipment of the vehicle, i.e. for example a car radio.

Additionally, the vehicle passenger(s) can also be warned of threatening health risks, for example of threatening cardiovascular problems. Therein it is particularly advantageous to determine the vital parameters of the vehicle passenger(s) for example of the vehicle driver, over longer periods of time, for example several days or weeks, and to analyze their progression, such that slow deteriorations can also be determined. In particular in the case of older passengers and/or passengers whose health is at risk or affected, in particular vehicle drivers, their sense of security and well-being is greatly increased. Additionally, in a medical emergency, an automatic emergency call can be made, wherein preferably the detected vital parameters can be transferred to an emergency call center or a hospital, such that quick and adequate medical help can be given. For this purpose, current positional date of the vehicle is preferably determined and transferred with the emergency call.

### BRIEF DESCRIPTION OF THE DRAWING FIGURES

Exemplary embodiments of the invention are illustrated in greater detail by means of drawings.

Here are shown:

FIG. 1 a schematic depiction of a vehicle with a vehicle passenger and an image capturing unit directed towards him,

FIG. 2 a schematic depiction of three color channels,

FIG. 3 a detailed view of a region from FIG. 2, and

FIG. 4 a schematic depiction of coefficients of a detected color signal.

Parts that correspond to one another have the same reference numerals added to them in all figures.

### DETAILED DESCRIPTION

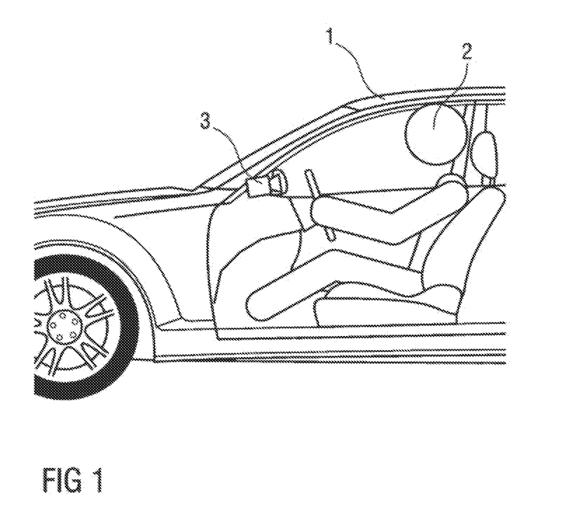

FIG. 1 shows a schematic depiction of a vehicle 1 with a vehicle passenger 2 and an image capturing unit 3 directed towards him as a device to monitor the vehicle passenger 2. In a method to monitor the passenger 2, images of the vehicle passenger 2 are captured by means of the image capturing unit 3, for example in the form of a video sequence, and are analyzed by means of an image processing unit, which is not depicted in more detail, in order to determine in this way vital parameters of the vehicle passenger 2, in particular a pulse rate, a breathing rate and a breathing volume, wherefrom a heart frequency variability is determined. The heart rate variability, also referred to as the HRV, is connected to the heart rate and the breathing i.e. the breathing rate and the breathing volume via the respiratory sinus arrhythmia, which describes a fluctuation of the heart rate in synchronization with the breathing. Additionally, vital functions or vital signs or health risks such as strength, stress tolerance, a cardiovascular risk and further health risks as well as positive and negative emotional states such as joy, stress or anger can be determined from the heart rate variability.

Furthermore, a skin temperature and/or pumping capacity, for example, of the heart and/or a blood pressure and/or an oxygen concentration in the blood of the vehicle passenger 2, also referred to as an oxygen saturation, and/or autonomous bodily functions, in particular functions of an autonomous nervous system of the vehicle passenger 2 and/or pain states of the vehicle passenger 2 can be determined. A vital state of the vehicle passenger 2 and a change of the vital state can be assessed by means of the determined vital parameters of the vehicle passenger 2.

For example, further diagnostic parameters are determined or recognized by means of the vital parameters, for example tachycardia, brachycardia and/or viral infections, by means of the heart rate variability, breathing, i.e. the breathing rate and/or the breathing volume, and/or the skin temperature. An assessment of the current vital state of the vehicle passenger 2 can be optimized by means of these further vital parameters, such that if necessary for example inattention of the vehicle passenger 2, for example of the vehicle driver, or a threatening or already occurring unconsciousness or severe health problems can be indicated.

The image capturing unit 3 is preferably an image capturing unit 3 that is already installed in the vehicle 1, for example, as is depicted here, in the region of the steering wheel, and is directed towards the vehicle passenger 2, in the example depicted here towards the vehicle driver. To carry out the monitoring method, a video camera with low resolution is sufficient therein, for example a so-called CCD camera. Special lighting devices are also not required; a normal environmental lighting in the vehicle 1, for example daylight, is already sufficient. The vital parameters of the vehicle passenger 2 are detected simultaneously by means of this image capturing unit 3 or all data is detected simultaneously from which the vital parameters of the vehicle passenger 2 can be determined by means of an image analysis.

A robustness of a signal detection of the image capturing unit 3 is clearly improved by partially redundant information, for example in an RGB color signal of the image capturing unit 3 and in particular by a possibility of the signal detection with a black and white camera for dark driving sections, i.e. for a dark environment, for example during a drive through a tunnel or at night. Therein, a signal detection in the so-called HSC black and white signal occurs, if necessary by means of a lighting by an infra-red light source. Therein, for example, a separate image detection unit 3 is not required, but the present image capturing unit 3 is switched to a black and white mode. Advantageously, this occurs automatically depending on respective proportions of environmental light.

The vital parameters are determined by detection of an optical absorption value and/or an intensity value of reflected light of a skin region of the vehicle passenger 2 and/or by determination of a change of this optical absorption value and/or of this intensity value. During a heartbeat, blood vessels, in particular arteries, expand slightly, as pressure increases in the blood vessels. This expansion causes an increase of the optical absorption and therefore also a decrease of the intensity of reflected light which is reflected by the skin region. Vital parameters of the vehicle passenger 2, in particular the pulse rate or heart rate and as well as the breathing rate and the breathing volume, can be determined by the detection of the differences in brightness of the light reflected by the skin region over time. This method is also called photoplethysmography. The foundations of the method are, for example, described in the article by Ming-Zher Poh, Daniel J. McDuff, and Rosalind W. Picard, “Noncontact, automated cardiac pulse measurements using video imaging and blind source separation,” Opt. Express 18 10762-10774 (2010), the complete content of which is included herein as a reference.

During the image processing, at least one region suitable for image analysis is searched for in each of the images by means of suitable methods and only this region is analyzed. This region is a region in the images, in which a sufficiently large skin region of the vehicle passenger 2 is displayed. Preferably a region of the images is searched for and analyzed as a region suitable for image analysis, which contains the face of the vehicle passenger 2, as in this region a sufficiently large skin region which is able to be analyzed is available and as, in particular, image detection units 3 which are already installed in the vehicle 1 and which for example are used to recognize alertness, as is also in the example depicted here, are directed towards the face of the vehicle passenger 2, in particular of the vehicle driver.

The location of such regions suitable for image evaluation is possible, for example by means of methods for facial recognition in images. One or several of the vital parameters of several vehicle passengers 2 can also be determined simultaneously if several vehicle passengers 2 are detected by the image capturing unit 3, wherein the image regions are determined, in which the faces of the vehicle passengers 2 are located and are analyzed separately from one another.

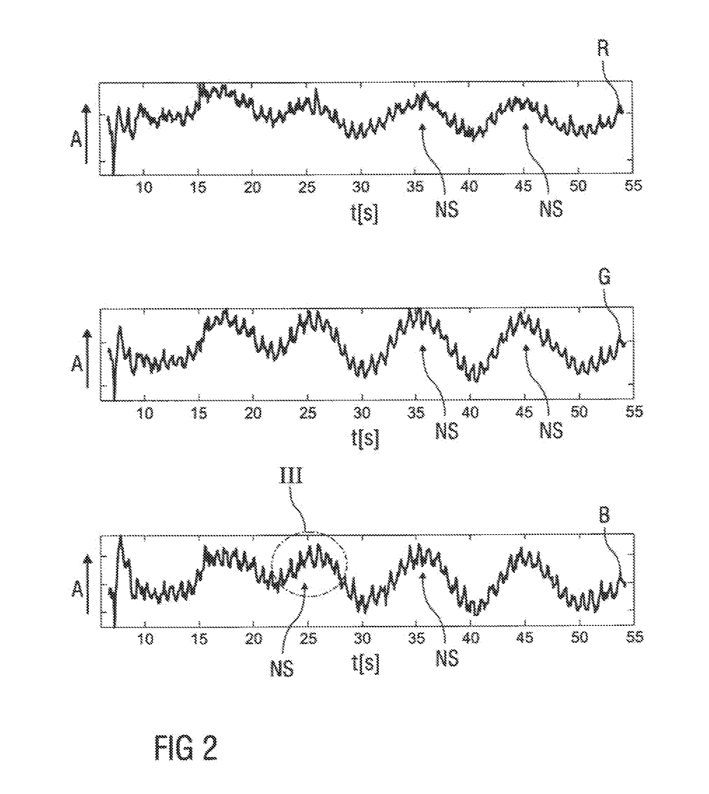

Advantageously, color images are captured as images of the vehicle passenger 2, in particular so-called RGB images, wherein the region of the images suitable for image analysis is split into individual color portions or color channels, in the case of RGB images into the corresponding RGB color channels, i.e. into a red color channel R, a green color channel G and blue color channel B, as is depicted in FIG. 2, and at least one color channel RGB is analyzed to determine the at least one vital parameter. Therein, preferably, in particular, the green portion, i.e. the green color channel G is analyzed, as in the case of this channel, the accuracy of the obtained results is the highest. In FIG. 2, these progressions of the color channels RGB are depicted over the time t.

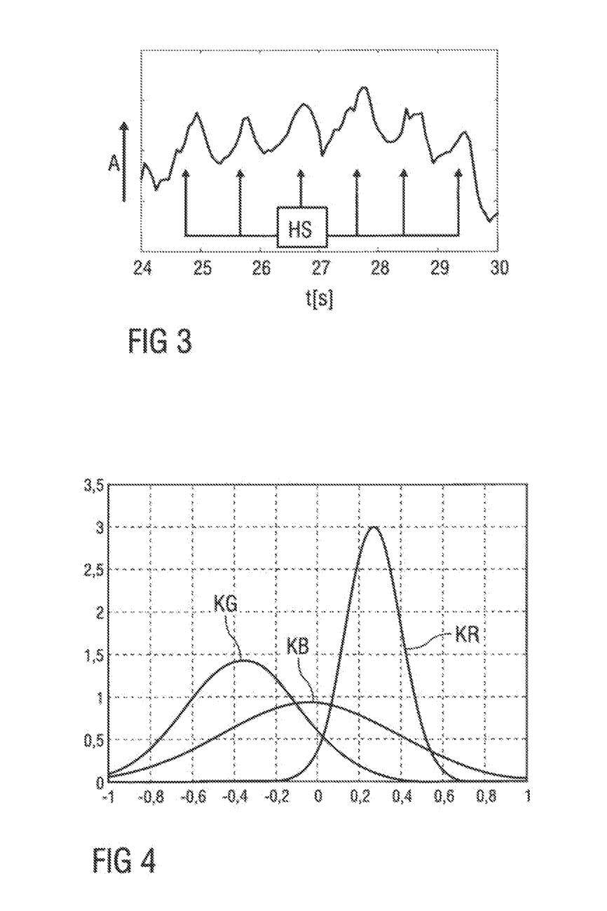

The breathing rate is determined by the analysis of the low-frequency vibrations NS of the color signal or color channels RGB, the breathing volume is determined by the analysis of the amplitudes A of the low-frequency vibrations NS and the heart rate is determined by the analysis of the high-frequency vibrations HS. In FIG. 3, the high-frequency vibrations HS of an individual low-frequency vibration of a color channel RGB is depicted.

A spectral power density of the color channel RGB to be analyzed of the region suitable for image analysis is determined by means of the fast Fourier transform. The differences in brightness of the light reflected by the skin region over time, and from this the vital parameter(s) of the vehicle passenger(s) 2, can then be determined by means of the determined spectral power density.

In FIG. 4, coefficients KR, KG, KB of the color signal split into the three color channels RGB of an area of skin of the vehicle passenger 2, for example of their face, is depicted. The skin temperature, the blood pressure and the oxygen saturation of the blood of the vehicle passenger 2 can be determined from the phase shift and/or the amplitude change between the coefficients KR, KG, KB of the RGB color signal.

The method is very simple and cost-effective to implement and carry out in the vehicle 1 due to the image capturing unit 3, which must only have a low resolution, as well as due to the implementation under normal environmental lighting conditions, i.e. without additional special light sources. Additionally, the method is very robust with respect to different skin colors as well as with respect to movements of the vehicle passengers(s) 2 during the image capturing. The determination of the vital parameters is therein sufficiently exact in order to be able to determine the vital state of the vehicle passenger(s) 2 sufficiently exactly, as well as a change of the vital state by means of the determined vital parameter(s), such that a vital state, which indicates for example an acute emergency situation and requires medical help or which for example could have effects on the safe driving of the vehicle 1, is able to be recognized with a high certainty.

The monitoring method or the vital parameter(s) of the vehicle passenger 2, in particular the vehicle driver, determined in this way can be used in a method to control at least one assistance device of the vehicle 1. Therein, at least one function of the assistance device is activated, if a determined value of at least one vital parameter deviates from a predetermined value or value region for this vital parameter.

In this way, the assistance device can intervene in a supporting manner in the case of poor or deteriorating vital parameters of the vehicle passenger(s) 2 in particular of the vehicle driver, in order, for example, to prevent an accident, to request help and/or to increase the comfort of the vehicle passenger(s) 2 in order to thus improve the vital state. This means the assistance device can, for example, be adapted to the current vehicle driver state or to the features of the vehicle driver, wherein these features, for example, can be evaluated by means of a stress level of the vehicle driver.

Preferably, an intervention in a steering device, a braking device and/or in a drive train of the vehicle 1 and/or an optical, aural and/or haptic warning device is activated as a function of the assistance device. In this way, the vehicle 1, as is described for example in German patent document DE 10 2009 016 936 A1, is steered to a roadside automatically and brought to a standstill there by a corresponding intervention in the steering device, braking device and/or the drive train of the vehicle 1. Particularly advantageously, the intervention in the steering device, the braking device and/or in the drive train of the vehicle 1 occurs in such a way that the vehicle 1 is autonomously steered to the next emergency assistance establishment, for example to a casualty of the nearest hospital.

Alternatively or additionally, warning devices of the vehicle 1 can be activated, for example outer warning devices in the form of a flashing warning light and/or a horn of the vehicle 1 in order to warn other road users, and/or warning devices in the vehicle interior can be activated in order to warn the vehicle passenger(s) 2, for example in order to wake a sleeping vehicle driver or to indicate to other vehicle passengers 2 a deteriorating vital parameter of the vehicle driver, which possibly indicates a deteriorated health state, such that his they can for example operate the steering wheel and/or the hand brake of the vehicle 1 in order to brake the vehicle 1 and/or to control it to the roadside.

Advantageously at least one comfort function and/or emergency call function is activated, alternatively or additionally, as a function of the assistance device. Through the activation of the comfort function or a plurality of such comfort functions, vital parameters of the vehicle passenger(s) can be improved, for example if the detected vital parameter(s) indicated fatigue, in particular of the vehicle driver.

Then, for example, a massage function of a vehicle seat, an interior ventilation and/or an air-conditioning function of the vehicle 1 can be activated. Therein essential oils or another refreshing medium can be introduced into the interior of the vehicle 1 to refresh the vehicle passenger(s) 2, for example. Furthermore, the current vital situation of the relevant passenger 2 can be indicated to him/her through optical, aural and/or haptic means, preferably combined with a recommendation of a suitable countermeasure, for example to take a restorative break, to move around regularly and/or to take on sufficient fluids. In this way, the vehicle passenger 2 can also be reminded to take required medication. Furthermore, for example, devices that support and/or calm the vehicle driver can be targetedly activated in the case of an increased stressed level recognized by means of the vital parameters, for example a navigation device or audio equipment of the vehicle 1 i.e. for example the car radio.

Additionally, the vehicle passengers 2 can also be warned of threatening health risks, for example of threatening cardiovascular problems. Therein, it is particularly advantageous to determine the vital parameters of the vehicle passenger(s) 2, for example of the vehicle driver, over longer periods of time, for example several days or weeks, and to analyze their progression, such that slow deteriorations can also be determined. In particular, in the case of older passengers 2 and/or passengers 2 who are at risk or affected in terms of health, in particular vehicle drivers, their sense of security and well-being is thus considerably improved. In a medical emergency, additionally, an automatic emergency call can be made, wherein preferably the detected and stored vital parameters are transferred to a emergency call center or to a hospital such that quick and adequate medical help can be given. For this purpose, current positional data of the vehicle 2 is also preferably determined and transferred with the emergency call.

The vital parameters determined by means of the monitoring method can also be combined with values of the vehicle passenger(s) 2 determined in another way, in order to obtain a better overall image of a state of the vehicle passenger(s) 2. In this way, for example, fatigue or inattention of the vehicle driver can be determined in good time and with a low error rate. Thus, for example, movements of the respective vehicle passenger 2, in particular of the vehicle driver, can be determined by sensors in the vehicle seat, which can indicate, restlessness, stress or the beginnings of fatigue. Additionally, the inattention recognition function, for which the image capturing unit 3 is originally installed in the vehicle 1, can also be used. In this way, determined additional values of the vehicle passenger(s) 2 can, for example, be combined with the determined pulse rate and/or with other determined vital parameters, in order to obtain a better estimation of the vital state of the vehicle passenger(s) 2.

The foregoing disclosure has been set forth merely to illustrate the invention and is not intended to be limiting. Since modifications of the disclosed embodiments incorporating the spirit and substance of the invention may occur to persons skilled in the art, the invention should be construed to include everything within the scope of the appended claims and equivalents thereof.

1-13. (canceled)

14. A method, comprising:
capturing, using at least one image capturing unit, images of a vehicle passenger in a vehicle; and
analyzing, by an image processing unit, the captured images to determine vital parameters of the vehicle passenger, wherein a pulse rate, a breathing rate and a breathing volume are determined as vital parameters and a heart rate variability is determined from the determined vital parameters.

15. The method according to claim 14, wherein further vital parameters with diagnostic value are determined from the vital parameters, wherein the further vital parameters include respiratory sinus arrhythmia or health risks including resilience, stress tolerance, a cardiovascular risk or emotional states.

16. The method according to claim 15, wherein the further vital parameters also include a skin temperature of the vehicle passenger, a pumping capacity of the heart of the vehicle passenger, a blood pressure or an oxygen concentration in blood of the vehicle passenger, autonomous bodily functions of the vehicle passenger, or pain states of the vehicle passenger.

17. The method according to claim 14, wherein further diagnostic quantities are determined by using the vital parameters, wherein the further diagnostic quantities include tachycardia, brachycardia, or viral infection.

18. The method according to claim 14, wherein at least one vital parameter is determined by a detecting an optical absorption value or an intensity value of reflected light of a skin region of the vehicle passenger, or by determining a change of the optical absorption value or of the intensity value.

19. The method according to claim 14, wherein, during the image processing, at least one region suitable for image analysis is searched for in each of the images and only the at least one region is analyzed.

20. The method according to claim 19, wherein the at least one region contains a face of the vehicle passenger.

21. The method according to claim 19, wherein color pictures are captured as the images of the vehicle passenger, wherein the at least one region is split into individual color channels and at least one color channel is analyzed to determine the vital parameters.

22. The method according to claim 19, wherein skin temperature, oxygen concentration, or oxygen saturation and blood pressure of the vehicle passenger is determined from a combination of the color channels based on a phase shift or amplitude change between the color channels.

23. The method according to claim 21, wherein a spectral power density of the color channel of the region suitable for image analysis is determined by using a fast Fourier transform.

24. The method of claim 14, further comprising:
activating at least one function of a assistance device if a determined value of at least one vital parameter deviates from a predetermined value or value region for the at least one vital parameter.

25. The method according to claim 24, wherein the activated at least one function is an intervention in a steering device of the vehicle, an intervention in braking device of the vehicle, an intervention in a drive train of the vehicle, an optical warning, aural warning, haptic warning, at least one comfort function, or an emergency call function.

26. A device to monitor at least one vehicle passenger in a vehicle, the device comprising:
at least one image capturing unit configured to capture images of the vehicle passenger;
at least one image processing unit configured to analyze the captured images, wherein the at least one image processing unit is configured to determine a pulse rate, a breathing rate and a breathing volume as vital parameters and determine a heart rate variability based on the determined vital parameters, and to determine a skin temperature of the vehicle passenger, a pumping capacity of a heart of the vehicle passenger, a blood pressure of the vehicle passenger, an oxygen concentration in blood of the vehicle passenger, autonomous bodily functions of the vehicle passenger, or pain states of the vehicle passenger.
